(19) United States
(12) Patent Application Publication
(10) Pub. No.: US 2025/0177036 A1
(43) Pub. Date: Jun. 5, 2025

(54) NEUROMODULATION CATHETER

(71) Applicant: Medtronic Ireland Manufacturing Unlimited Company, Dublin (IE)

(72) Inventors: Syamala Rani Pulugurtha, Irvine, CA (US); Ujwal Jalgaonkar, Irvine, CA (US); Paul J. Coates, Corte Madera, CA (US)

(21) Appl. No.: 18/841,996

(22) PCT Filed: Feb. 22, 2023
(86) PCT No.: PCT/EP2023/054444

§ 371 Date: Aug. 27, 2024

Related U.S. Application Data
(60) Provisional application No. 63/268,687, filed on Feb. 28, 2022.

(51) Int. Cl. A61B 18/14; A61B 18/00; A61B 90/00

(52) U.S. Cl. CPC A61B 18/1492; A61B 90/39; A61B 2018/00077; A61B 2018/00184; A61B 2018/00434; A61B 2090/3966

(57) ABSTRACT

An example catheter includes an elongate shaft including a proximal portion and a distal portion including a neuromodulation element. The neuromodulation element includes a plurality of electrodes disposed along a length of an elongate structure and a bend between a first segment of the length of the elongate structure and second segment of the length of the elongate structure such that the second segment extends in a proximal direction from the bend.

### TECHNICAL FIELD

The present technology is related to catheters, such as, for example, neuromodulation catheters including a neuromodulation element configured to deliver energy to nerves at or near a treatment location within a body lumen.

### BACKGROUND

Catheters including one or more energy delivery elements have been proposed for use in various medical procedures, including neuromodulation procedures. For example, some catheters include a plurality of electrodes configured to deliver radiofrequency energy to a region of tissue during an ablation procedure.

### SUMMARY

The present disclosure is directed to catheters and techniques for making and using the same. Some examples are directed to catheters and techniques for neuromodulation (e.g., denervation), such as renal neuromodulation. In some examples, a catheter may be configured to deliver RF energy circumferentially around a lumen of the patient in which the catheter is positioned. For example, the lumen may include a blood vessel such as a renal main artery, an accessory renal artery, or a branch vessel, for non-renal-nerve neuromodulation, or a body lumen other than a vessel, for extravascular neuromodulation, and/or for use in therapies other than neuromodulation.

The catheter may include at least a proximal portion and a distal portion. The distal portion may include a neuromodulation element including a plurality of electrodes. The distal portion of the catheter may be configured to transform between a radially compressed delivery configuration and an expanded deployed configuration. The distal portion of the catheter may include a bend such that a first segment of the length of the distal portion extends in the proximal direction, e.g., a portion of the length of the distal portion may be folded back on itself via a bend or cylindrical bend, or fold, or the like. In some examples, the distal portion may include a plurality of bends such that a plurality of segments extend alternatingly in the proximal and distal directions. In some examples, one or more of the plurality of bends may be such that the subsequent segment is out-of-plane with a plane including the previous two segments.

In some examples, at least two of the plurality of electrodes may be configured to be positioned at a first longitudinal position, e.g., the same longitudinal position, along the distal portion. The at least two of the plurality of electrodes maybe configured to provide a neuromodulation treatment to tissue of a patient at different circumferential positions within the vasculature of the patient at the first longitudinal position, e.g., within substantially the same circumferential plane about the first longitudinal position.

In one example, this disclosure describes a catheter including an elongate shaft including a proximal portion and a distal portion including a neuromodulation element, wherein the neuromodulation element comprises: a plurality of electrodes disposed along a length of an elongate structure; and a bend between a first segment of the length of the elongate structure and second segment of the length of the elongate structure such that the second segment extends in a proximal direction from the bend.

In another example, this disclosure describes a method including navigating a catheter through vasculature of a patient to a target treatment site in a vessel of the patient, wherein the catheter comprises an elongate shaft including a proximal portion and a distal portion including a neuromodulation element, wherein the neuromodulation element comprises: a plurality of electrodes disposed along a length of an elongate structure; and a bend between a first segment of the length of the elongate structure and second segment of the length of the elongate structure such that the second segment extends in a proximal direction from the bend.

In another example, this disclosure describes a method including disposing a plurality of electrodes along a length of an elongate structure of a neuromodulation element of distal portion of an elongate shaft of a catheter; and bending the elongate structure to form a bend such that a first segment of the length of the elongate structure extends between the bend and the elongate shaft and a second segment of the length of the elongate structure extends in a proximal direction from the bend.

Further disclosed herein is a catheter that includes an elongate shaft including a proximal portion and a distal portion including a neuromodulation element, wherein the neuromodulation element includes a plurality of electrodes disposed along a length of an elongate structure and a bend between a first segment of the length of the elongate structure and second segment of the length of the elongate structure such that the second segment extends in a proximal direction from the bend.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Reference is made to the attached drawings, wherein elements having the same or similar reference numeral designations represent similar elements throughout.

FIG. 1 is a partially schematic perspective view illustrating a therapeutic system configured in accordance with an example of the present technology. The system shown in FIG. 1 includes a neuromodulation catheter having a shaft.

FIG. 2 is a profile view of the catheter shown in FIG. 1.

FIG. 3A is an enlarged profile view of portions of an example neuromodulation element taken at respective locations designated in FIG. 2 and in a deployed configuration.

FIG. 3B is another enlarged profile view of portions of the example neuromodulation element of FIG. 3A taken at respective locations designated in FIG. 2 and in a deployed configuration.

FIG. 3C is an enlarged profile view of portions of another example neuromodulation element taken at respective locations designated in FIG. 2 and in a deployed configuration.

FIG. 4A is a cut-away of the view of an enlarged profile view of portions of another example neuromodulation in a deployed configuration.

FIG. 4B is a cross-sectional view of the example neuromodulation element of FIG. 4A taken along a longitudinal position indicated in FIG. 4A and in a deployed configuration within a vessel.

FIG. 5 is a cut-away of the view of an enlarged profile view of portions of another example neuromodulation element in a deployed configuration.

FIG. 6 is a cut-away of the view of an enlarged profile view of portions of another example neuromodulation element in a deployed configuration.

FIG. 7 is a cut-away of the view of an enlarged profile view of portions of another example neuromodulation element in a deployed configuration.

FIG. 8 is a cut-away of the view of an enlarged profile view of portions of another example neuromodulation element in a deployed configuration.

FIG. 9 is a cut-away of the view of an enlarged profile view of portions of another example neuromodulation element in a deployed configuration.

FIG. 10 is a cut-away of the view of an enlarged profile view of portions of another example neuromodulation element in a deployed configuration.

FIG. 11 is a flow chart illustrating an example method for using a neuromodulation catheter, in accordance with one or more examples of the present disclosure

FIG. 12 is a flow chart illustrating an example method for assembling components of a neuromodulation catheter, in accordance with one or more examples of the present disclosure.

### DETAILED DESCRIPTION

The present technology is directed to neuromodulation catheters and techniques for assembling a neuromodulation catheter. Although the systems, devices, and methods may be disclosed herein primarily or entirely with respect to intravascular renal neuromodulation, other applications in addition to those disclosed herein are within the scope of the present technology. For example, systems, devices, and methods in accordance with at least some examples of the present technology may be useful for neuromodulation within a body lumen other than a vessel, for extravascular neuromodulation, for non-renal neuromodulation, and/or for use in therapies other than neuromodulation. The present technology may be directed to, for example, renal neuromodulation, spinal neuromodulation, cardiac neuromodulation, brain neuromodulation, sacral neuromodulation, urinary neuromodulation, and/or neuromodulation techniques directed to other portions of a body. A catheter may be configured (e.g., have suitable shape and dimensions) to deliver energy (e.g., radiofrequency, pulsed field, direct electrical current) with a portion of the catheter carrying an electrode positioned in tissue or in an anatomical lumen (e.g., a renal artery, external iliac artery, internal iliac artery, internal pudendal artery, celiac artery, mesenteric artery, superior mesenteric artery, inferior mesenteric artery, hepatic artery, splenic artery, gastric artery, left gastric artery, pancreatic artery, uterine artery, ovarian artery, testicular artery, and/or their associated arterial branches, accessories, veins, etc. and/or other anatomical lumens). Although examples are described primarily with respect to renal neuromodulation, a person having ordinary skill in the art reading this description will understand that the devices, systems, and methods described herein may be used for neuromodulation at any suitable location within a body of a patient, including intravascular locations. Exemplifying vascular locations include, for example, a renal artery, external iliac artery, internal iliac artery, internal pudendal artery, celiac artery, mesenteric artery, superior mesenteric artery, inferior mesenteric artery, hepatic artery, splenic artery, gastric artery, left gastric artery, pancreatic artery, uterine artery, ovarian artery, testicular artery, and/or their associated arterial branches, accessories, veins, and the like. Furthermore, it should be understood, in general, that other systems, devices, and methods in addition to those disclosed herein are within the scope of the present technology. For example, systems, devices, and methods in accordance with examples of the present technology can have different and/or additional configurations, components, and procedures than those disclosed herein. Moreover, a person of ordinary skill in the art will understand that systems, devices, and methods in accordance with examples of the present technology can be without one or more of the configurations, components, and/or procedures disclosed herein without deviating from the present technology.

As used herein, the terms “distal” and “proximal” define a position or direction with respect to the treating clinician or clinician's control device (e.g., a handle assembly).

“Distal” or “distally” can refer to a position distant from or in a direction away from the clinician or clinician's control device. “Proximal” and “proximally” can refer to a position near or in a direction toward the clinician or clinician's control device.

Neuromodulation, such as denervation, may be used to modulate activity of one or more nerves and may be used to affect activity of the sympathetic nervous system (SNS). Renal neuromodulation, for example, may be used to modulate activity of one or more renal nerves and may be used to affect activity of the SNS. In renal neuromodulation, one or more therapeutic elements may be introduced near renal nerves located between an aorta and a kidney of a patient. In some examples, the one or more therapeutic elements may be carried by or attached to a catheter, and the catheter may be introduced intravascularly, e.g., into a renal artery via a brachial artery, femoral artery, or radial artery approach. In other examples, the one or more therapeutic elements may be introduced extravascularly, e.g., using a laparoscopic technique.

Neuromodulation may be accomplished using one or more of a variety of treatment modalities, including electrical stimulation, radio frequency (RF) energy, microwave energy, ultrasound energy, a chemical agent, thermal energy (e.g., cryoablation or direct heating) or the like. Although examples are described primarily with respect to the RF modality, a person having ordinary skill in the art reading this description will understand that the devices, systems, and methods described herein also may be used for neuromodulation employing other modalities, including those referenced herein. In some examples, an RF ablation system includes an RF generator configured to generate RF energy and deliver RF energy to tissue via a plurality of electrodes carried by a catheter and positioned within a lumen of a body of a patient. For example, the lumen may be a vessel, such as a vein or artery. In some examples, the lumen may be a renal artery, such as a main renal artery, an accessory renal artery, a branch vessel, or the like. The RF energy may heat tissue to which the RF energy is directed (which tissue includes one or more renal nerves) and modulate the activity of the one or more renal nerves.

In many patients, renal nerves generally follow the renal artery and branch vessels from near the aorta to a kidney. The renal nerves may be present in a wall of the renal artery and/or branch vessels and/or in tissue surrounding the renal artery and/or branch vessels. Because renal nerves may be circumferentially around the renal artery and/or branch vessels and may include multiple nerves and/or nerve branches, it may be desirable to deliver RF energy circumferentially around the renal artery and/or branch vessels to affect (e.g., modulate, treat, denervate, and the like) as many renal nerves as possible. It may be desirable to deliver RF energy circumferentially around the renal artery and/or branch vessels at substantially the same longitudinal position, e.g., in the same circumferential plane, to affect renal nerves that may change circumferential position as a function of longitudinal position along the artery and/or branch vessels. In other words, in order to deliver RF energy to renal nerves which may wind around the artery and/or branch vessels, it many be desirable to deliver RF energy at different circumferential positions of a vessel or other lumen at the same longitudinal position in the vessel or other lumen, e.g., at circumferentially opposite sides of the artery and/or branch vessels, or at multiple circumferential positions of the artery and/or branch vessels.

In accordance with examples of the current disclosure, a catheter (e.g., an RF ablation catheter) may include a neuromodulation element that is configured to deliver RF energy circumferentially around a lumen of the patient (e.g., a blood vessel such as a renal main artery, accessory renal artery, or branch vessel, or a body lumen other than a vessel) in which a distal portion of the catheter is positioned. For example, a catheter may include at least a proximal portion and a distal portion. The distal portion may include a neuromodulation element including a plurality of electrodes (e.g., two electrodes, three electrodes, four electrodes, or the like) and may be configured to transform between a compressed delivery configuration (e.g., to position the neuromodulation element within a lumen of the patient) and an expanded deployed configuration in which the electrodes are configured to deliver RF energy at different circumferential positions at substantially the same longitudinal position of a lumen. In both the delivery and deployed configuration, the neuromodulation element of the distal portion may including one or more segments that bend and/or fold back in generally the opposite direction from the previous segments, e.g., so as to position electrodes at a plurality of circumferential positions at substantially the same longitudinal position along the distal portion.

In such a configuration, it may be difficult to utilize a guidewire within a lumen of the catheter to advance and/or retract the catheter, e.g., because of a tortuous path including the one or more bends of the distal portion. In accordance with examples of the current disclosure, the distal portion may including one or more guidewire ports configured to receive a guidewire, e.g., within a lumen of the catheter and/or distal portion. For example, the one or more guidewire ports may facilitate advancing and/or retracting the catheter via the guidewire with the guidewire being within the lumen of a portion of the length of the distal portion, e.g., within the lumen of one of the segments of the distal portion.

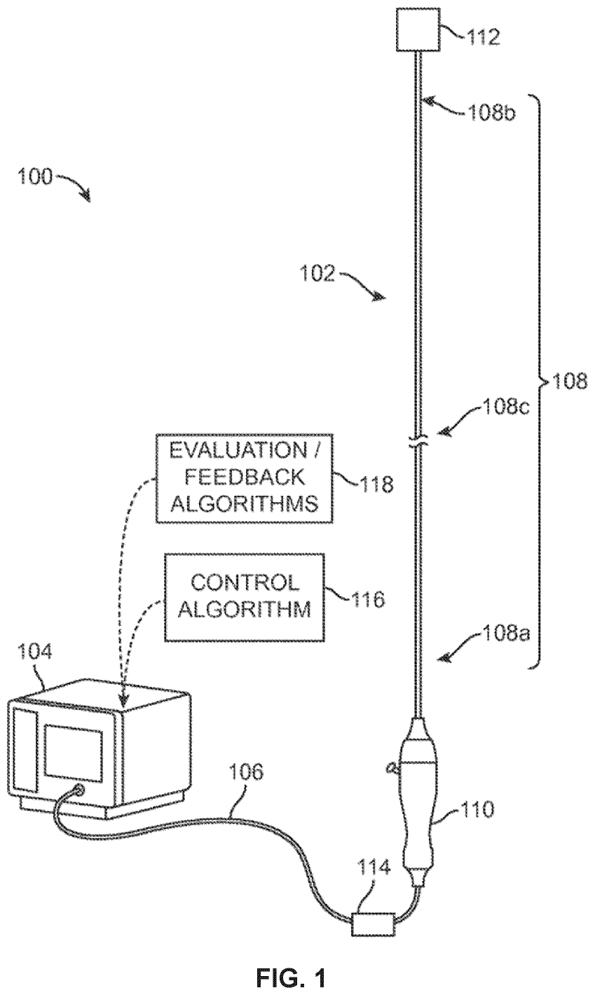

FIG. 1 is a partially schematic perspective view illustrating a therapeutic system 100 including a neuromodulation catheter 102 configured in accordance with some examples of the present disclosure. Therapeutic system 100 includes a neuromodulation catheter 102, an RF generator 104, and a cable 106 extending between catheter 102 and RF generator 104. Neuromodulation catheter 102 includes an elongate shaft 108 (also referred to as an elongate body 108) having a proximal portion 108a, a distal portion 108b, and an intermediate portion 108c between proximal portion 108a and distal portion 108b. Neuromodulation catheter 102 may further include a handle 110 (e.g., a proximal assembly) operably connected to elongate shaft 108 via proximal portion 108a and a neuromodulation element 112 (shown schematically in FIG. 1) that is part of or attached to distal portion 108b. Elongate shaft 108 is configured to locate the neuromodulation element 112 (e.g., a distal assembly) at a treatment location within or otherwise proximate to a body lumen (e.g., a blood vessel, a duct, an airway, or another naturally occurring lumen within the human body). In some examples, elongate shaft 108 is configured to locate neuromodulation element 112 at an intraluminal (e.g., intravascular) location. Neuromodulation element 112 may be configured to provide or support a neuromodulation treatment at the treatment location. Elongate shaft 108 and neuromodulation element 112 may measure 2, 3, 4, 5, 6, or 7 French or other suitable sizes.

Intraluminal delivery of neuromodulation catheter 102 may include percutaneously inserting a guidewire (not shown) into a body lumen of a patient and moving elongate shaft 108 and neuromodulation element 112 (e.g., a distal assembly) along the guidewire until neuromodulation element 112 reaches a suitable treatment location. Alternatively, neuromodulation catheter 102 may be a steerable or non-steerable device configured for use without a guidewire. Additionally, or alternatively, neuromodulation catheter 102 may be configured for use with another type of guide member, such as a guide catheter or a sheath (not shown), alone, or in addition to a guidewire.

RF generator 104 is configured to control, monitor, supply, and/or otherwise support operation of neuromodulation catheter 102. In other examples, neuromodulation catheter 102 may be self-contained or otherwise configured for operation independent of RF generator 104. When present, RF generator 104 is configured to generate a selected form and/or magnitude of RF energy for delivery to tissue at a treatment location via neuromodulation element 112. In some examples, RF generator 104 may be configured to generate RF energy (e.g., monopolar and/or multipolar (e.g., bipolar) RF energy). For example, RF generator 104 may be configure to generate RF energy in a monopolar configuration in conjunction with a return electrode placed on the patient's skin, or in a bipolar configuration in which the RF energy is delivered between electrodes of neuromodulation catheter 102. In other examples, RF generator 104 may be another type of device configured to generate and deliver another suitable type of energy to neuromodulation element 112 for delivery to tissue at a treatment location via neuromodulation element 112.

Along cable 106 or at another suitable location within therapeutic system 100, therapeutic system 100 may include a control device 114 configured to initiate, terminate, and/or adjust operation of one or more components of neuromodulation catheter 102 directly and/or via RF generator 104. In some examples, control device 114 may be a part of, or located on, RF generator 104, e.g., including a button, dial, or other control feature on RF generator 104. In other examples, control device 114 may be a separate component similar to control device 114 as shown in the example except as a separate control device 114 connected to RF generator 104 rather than connected to both RF generator 104 and handle 10 as shown. RF generator 104 may be configured to execute an automated control algorithm 116 and/or to receive control instructions from an operator. Similarly, in some implementations, RF generator 104 is configured to provide feedback to an operator before, during, and/or after a treatment procedure via an evaluation/feedback algorithm 118.

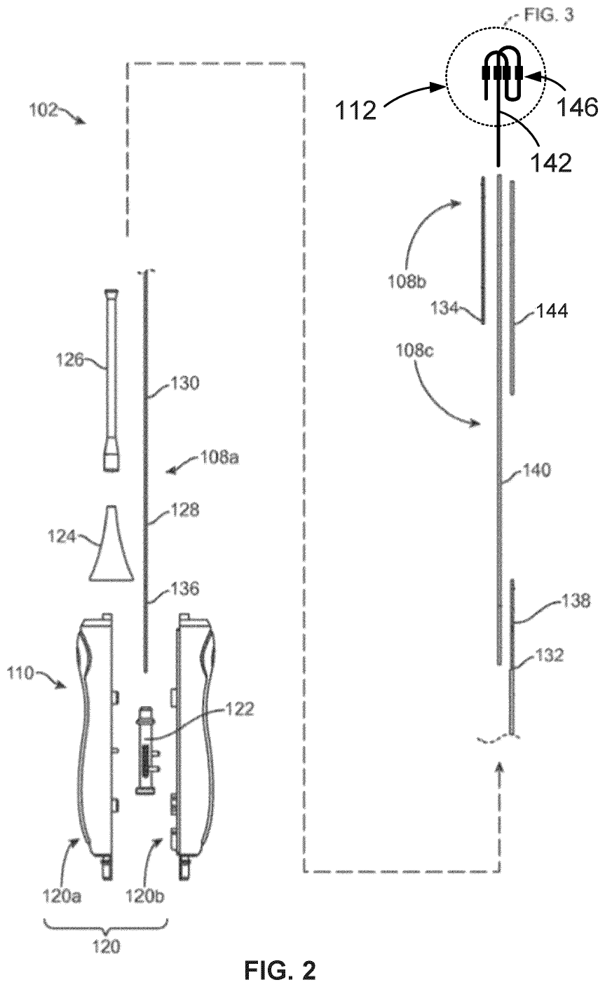

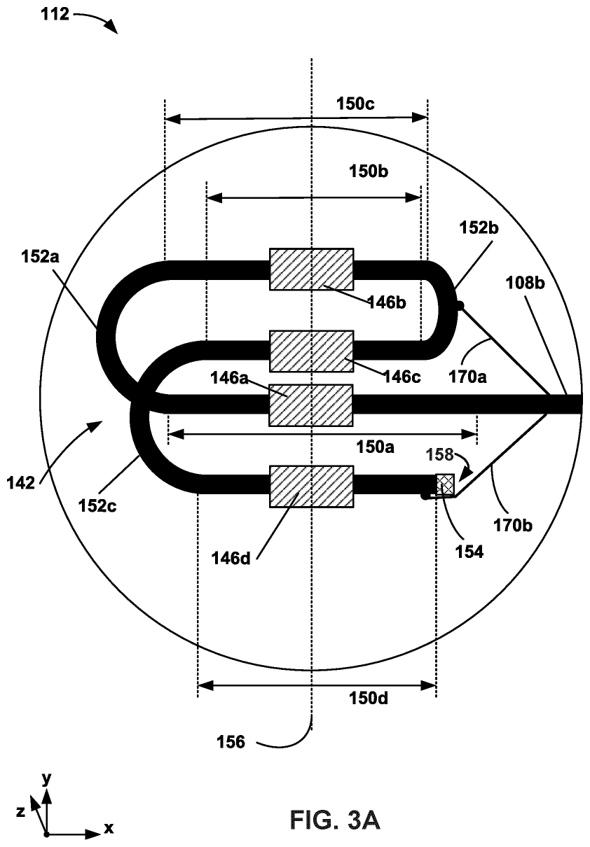

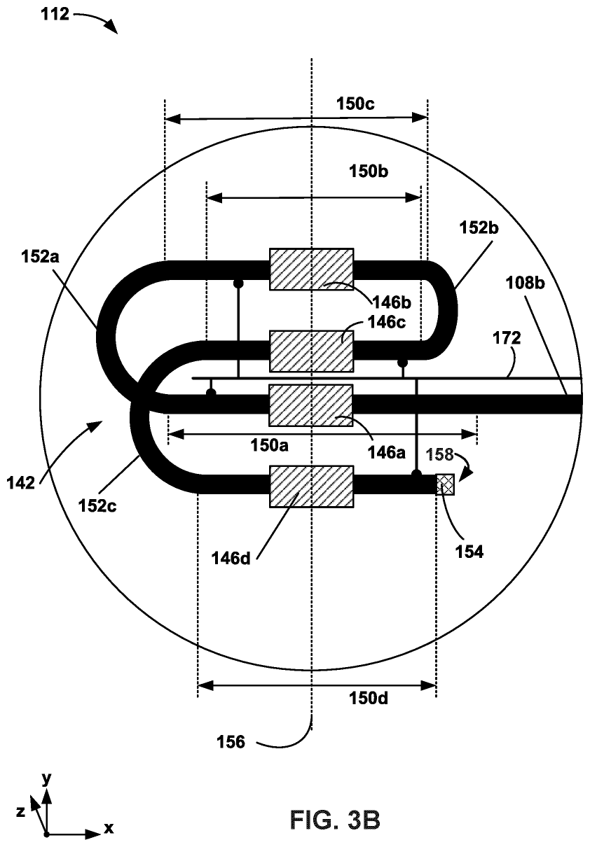

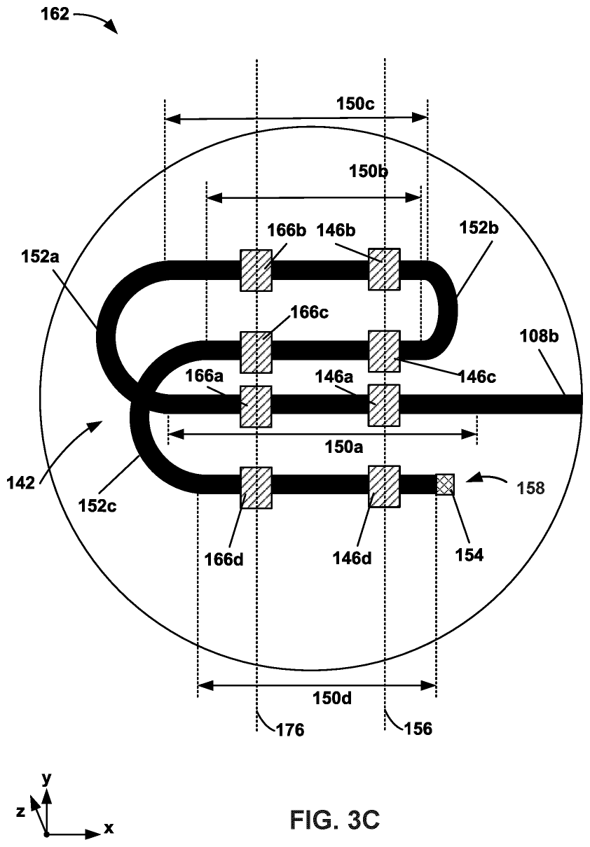

FIG. 2 is an profile view of neuromodulation catheter 102. FIG. 3A is an enlarged profile view of portions of neuromodulation catheter 102 taken at the location designated in FIG. 2 and in a deployed configuration. FIG. 3B is an enlarged profile view of portions of neuromodulation catheter 102 taken at respective locations designated in FIG. 2 and in a deployed configuration illustrating another example neuromodulation element 162.

With reference to FIG. 2, handle 110 may include mating shell segments 120 (individually identified as shell segments 120a, 120b) and a connector 122 (e.g., a luer connector) operably positioned between the mating shell segments 120. Handle 110 may further include a distally tapered strain-relief element 124 operably connected to distal ends of shell segments 120. Slidably positioned over elongate shaft 108, neuromodulation catheter 102 may include a loading tool 126 configured to facilitate loading catheter 102 onto a guidewire (not shown).

Elongate shaft 108 may include an assembly of parallel tubular segments. At proximal portion 108a, elongate shaft 108 may include a proximal hypotube segment 128, a proximal outer jacket 130, and a first electrically insulative tube 132. Proximal outer jacket 130 may be a polymer outer jacket disposed around at least a portion of an outer surface of proximal hypotube segment 128. For example, proximal outer jacket may include a polyether block amide (e.g., PEBAX® 7233, PEBAX® 7233 with twenty percent weight siloxane, or the like). First electrically insulative tube 132 may be an electrically insulative polymer tube or coating located at least partially within an inner lumen of proximal hypotube segment 128. First electrically insulative tube 132 may be attached to proximal hypotube segment 128 at at least one of a proximal portion or a distal portion of proximal hypotube segment 128. First electrically insulative tube 132 may define an inner lumen, and one or more electrical leads (e.g., electrically conductive wires) may be positioned within the inner lumen of first electrically insulative tube 132. First electrically insulative tube 132 may electrically insulate the electrical leads from proximal hypotube segment 128. The one or more electrical leads may extend distally within the inner lumen of proximal hypotube segment 128 and electrically insulative tube 132 to neuromodulation element 112 on the distal portion of the elongate shaft (e.g., to one or more of electrodes 146a, 146b, 146, 146c, collectively “electrodes 146,” of neuromodulation element 112).

Proximal hypotube segment 128 may include a proximal portion, a proximal end, and a first inner lumen. When assembled, handle 110 (e.g., proximal assembly) may define a second inner lumen, wherein the proximal portion of proximal hypotube segment 128 is received within the second inner lumen of the handle 110, and the proximal end of proximal hypotube segment 128 terminates within the second inner lumen. For example, when assembled, elongate shaft 108 may extend through coaxial lumens (also not shown) of strain-relief element 124 and loading tool 126, respectively, and between shell segments 120 to connector 122. Proximal hypotube segment 128 may define a length. In some examples, the length may be longer or shorter to allow for different types of access to a patient's body. In some examples, proximal hypotube segment 128 and/or elongate shaft 108 may be longer for radial access to a patient's body than proximal hypotube segment 128 and/or elongate shaft 108 for femoral access to a patient's body. In some examples of an elongate shaft 108 for femoral access to a patient's body, a combined length of proximal hypotube segment 128 and an intermediate segment of elongate shaft 108 may be around 32 inches long. In some examples of an elongate shaft 108 for radial access to a patient's body, a combined length of proximal hypotube segment 128 and an intermediate segment of elongate shaft 108 may be around 56 inches long. Proximal hypotube segment 128 and the intermediate segment may be made predominantly from nitinol, stainless steel, or other suitable materials.

Elongate shaft 108 may include an intermediate segment 140 beginning proximally at a region of elongate shaft 108 at which first electrically insulative tube 132 distally emerges from proximal hypotube segment 128. Intermediate segment 140 may be more flexible than proximal hypotube segment 128. Intermediate segment 140 may be attached at one end to the distal assembly (e.g., neuromodulation element 112) and attached at the other end to proximal hypotube segment 128 via a rapid exchange joint. Intermediate segment 140 may be coaxially aligned with proximal hypotube segment 128 so that a rapid exchange joint may be formed. From this region, intermediate segment 140 may extend distally to distal end portion 108b of shaft 108. At a distal end of intermediate segment 140, elongate shaft 108 may be operably connected to neuromodulation element 112 (e.g., a distal assembly). Intermediate segment 140 may be attached to the distal assembly or proximal hypotube segment 128 using reflowed polymer, an adhesive, or the like. For example, intermediate segment 140 may be attached to the distal assembly or proximal hypotube segment 128 using a polyether block amide (e.g., PEBAX®, commercially available from Arkema Group of Colombes, France). In addition to, or instead of, PEBAX®, intermediate segment may be attached to the distal assembly or proximal hypotube segment 128 using another reflowable polymer. The one or more electrical leads may extend distally within an inner lumen of intermediate segment 140 to neuromodulation element 112 on distal portion 108b of elongate shaft 108 (e.g., to electrodes 146 of neuromodulation element 112).

A proximal end of guidewire tube 134 may be positioned in the rapid exchange joint to allow a guidewire to pass from the inside of elongate shaft 108 distal to the rapid exchange joint to the outside of elongate shaft 108 proximal of the rapid exchange joint. A proximal portion of guidewire tube 134, including the proximal end of guidewire tube 134 may be attached to elongate shaft 108 using reflowed polymer, an adhesive, or the like. The proximal portion of guidewire tube 134 may include a skived section configured to define a smooth diameter transition between proximal outer jacket 130 (e.g., a polymer outer jacket) and a location where a portion of guidewire tube 134 extends into the rapid exchange joint. The guidewire may be positioned inside an interior lumen of guidewire tube 134 in a distal portion 108b of elongate shaft 108, exit elongate shaft 108 at the rapid exchange joint, and be positioned outside elongate shaft 108 from the rapid exchange joint proximally of the rapid exchange joint (e.g., to at least handle 110). Guidewire tube 134 may extend distally from the rapid exchange joint to neuromodulation element 112. In some examples, guidewire tube 134 may extend from neuromodulation element 112 to an exit port on catheter 102 that is on the outside of elongate body 108, e.g., an “over-the-wire” configuration.

In FIGS. 2-4A and 5-10, neuromodulation element 112 is shown in a radially expanded deployed state. Neuromodulation element 112 may be movable from a low-profile delivery state to the radially expanded deployed state. When neuromodulation element 112 is in the radially expanded deployed state, an elongate structure 142 may have a decompressed and/or relaxed shape. When neuromodulation element 112 is in the low-profile delivery state, elongate structure 142 may have a compressed and/or reduced radial profile shape. Elongate structure 142 may comprise a shape-memory material or structure, e.g., a Nitinol-based structure (such as a helical hollow strand (HHS®) structure, available from Fort Wayne Metals, Fort Wayne, Indiana), or another shape-memory material.

In at least some cases, elongate structure 142 has an expanded, e.g., radially expanded, shape when at rest and is configured to be urged into the compressed shape by an external sheath (not shown), an internal guidewire, an internal mandrel, or the like. For example, referring to FIG. 3A, elongate structure 142 includes one or more features 170 (e.g., 170a and 170b in the example shown) configured to urge a proximal end of elongate structure 142 to collapse to a smaller, more capturable diameter. For example, elongate structure 142 may be proximally drawn into an external sheath (not shown, or alternatively the sheath may be distally advanced toward elongate structure 142). A distal end and/or opening of the sheath may contact the features 170. The features 170 may be connected to portions of elongate structure 142, e.g., segment 150d near distal tip 154 and a proximal facing portion of bend 152b in the example shown, and distal portion 108b. The features 170 may be controlled, e.g., features 170 may be wires manipulable by a user and/or clinician, to pull at least a proximal portion of elongate structure 142 to be radially compressed and to be received within the sheath, or the distal end of the sheath may contact features 170 causing the features 170 to pull on proximal portions of elongate structure 142 to radially compress proximal portions of elongate structure 142 to be received within the sheath. FIG. 3B is an enlarged profile view of portions of the example elongate structure 142 of FIG. 3A illustrating alternate feature 172. In the example shown, feature 172 may be connected to one or more portions of elongate structure 142, e.g., one or more of segments 150 as in the example shown. In some examples, feature 172 is a pull wire configured to reduce the diameter and/or radial profile shape of elongate structure 142 to be received with the sheath, e.g., a pull wire manipulable such as by a user and/or clinician.

In some examples, elongate structure may be a portion of distal portion 108b, e.g., made of the same material and/or integral to distal portion 108b. In other examples, elongate structure 142 may comprise a different material from distal portion 108b and may be attached to or integral to distal portion 108b. In at least some cases, the material of elongate structure 142 is electrically conductive. Accordingly, neuromodulation element 112 may include a second electrically insulative tube 144 disposed around an outer surface of elongate structure 142 so as to electrically separate electrodes 146 from elongate structure 142. In some examples, first and second electrically insulative tubes 132, 144 are made at least partially (e.g., predominantly or entirely) of polyimide and polyether block amide, respectively. In other examples, first and second electrically insulative tubes 132, 144 may be made of other suitable materials, e.g., polyurethane, polyester, other suitable polymers or predominantly polymer materials. A distal jacket (not shown) may be tubular and configured to be disposed around at least a portion of an outer surface of elongate structure 142, e.g., at neuromodulation element 112.

Neuromodulation element 112 may be configured to transform between a substantially compressed delivery configuration and a deployed configuration (e.g., an expanded or radially expanded configuration). Neuromodulation catheter 102 may further include one or more wires (not shown in FIGS. 2 and 3) or wire pairs extending from a proximal end (or from near the proximal end) of neuromodulation catheter 102 to electrode(s) 146 at distal portion 108b of neuromodulation catheter 102, each wire (or wire pair) of one or more wires being electrically coupled (e.g., welded or otherwise affixed) to a corresponding electrode of electrodes 146 to deliver energy, and in some examples, to form a thermocouple for conducting temperature measurements. Distal portion 108b of neuromodulation catheter 102 may also define one or more openings or slots through which the one or more wire(s) extend in order to contact and electrically couple to the one or more electrode(s) 146.

In some examples, neuromodulation element 112 may include one or more bends of elongate structure 142. In the example shown in FIG. 3A, neuromodulation element 112 includes first bend 152a, second bend 152b, and third bend 152c (collectively “bends 152”) along the length of elongate structure 142. In such a configuration, elongate structure 142 of neuromodulation element 112 may include one or more segments, e.g., extending from and/or to bends 152. In the example shown, neuromodulation element includes segments 150a, 150b, 150c, and 150d, collectively “segments 150.” Segment 150a extends between distal portion 108b and first bend 152a, segment 150b extends in a proximal direction from first bend 152a. For example, elongate structure 142 may be bent and/or folded, e.g., via a cylindrical bend or any other suitable bend or fold type and/or bend or fold method, to form segment 150a, first bend 152a, and segment 150b. In the example shown, elongate structure 142 may be further bent and/or folded to form second bend 152b, segment 150c, third bend 152c, and segment 150d. Electrodes 146 may be positioned along respective segments 150 such that electrodes 146 are disposed substantially at longitudinal position 156 when neuromodulation element 112 is in the deployed configuration, e.g., as shown. In other examples, neuromodulation element 112 may include more or fewer segments 150 and bends 152, and more or fewer electrodes 146. In some examples, segments 150 may be configured to bias electrodes 146 towards, or away, from a vessel wall. For example, segments 150 may include a gradual bend and or curvature, at least in the deployed configuration, which may radially extend electrodes 146 towards the vessel (or other body lumen) wall and may increase contact and/or pressure of the electrode against the vessel wall, e.g., relative to a substantially straight segment 150. In other examples, segments 150 may include a gradual bend and or curvature, at least in the deployed configuration, which may radially extend electrodes 146 away from the vessel (or other body lumen) wall and may be configured to position electrodes 146 near, but with no and/or decreased contact with the vessel wall, e.g., to reduce heat transfer to tissue during delivery of RF energy, denervation, modulation of nerves, or the like.

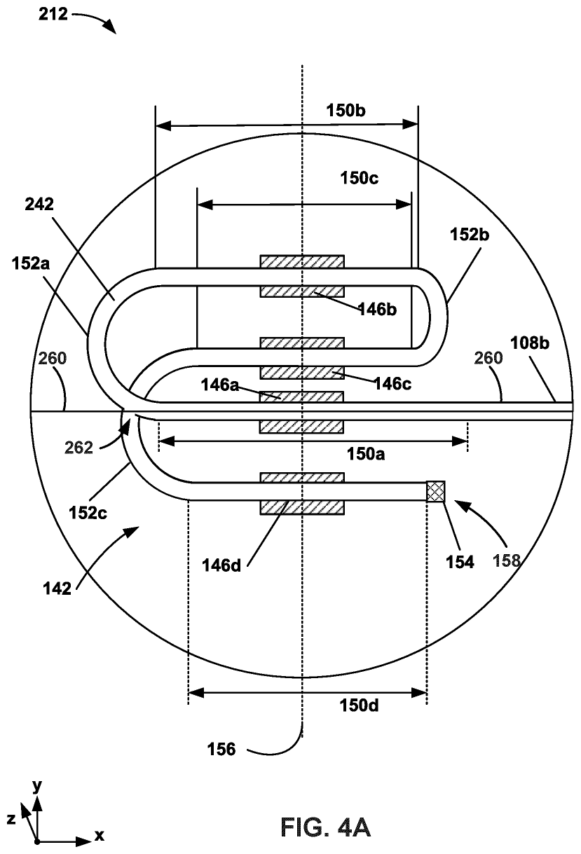

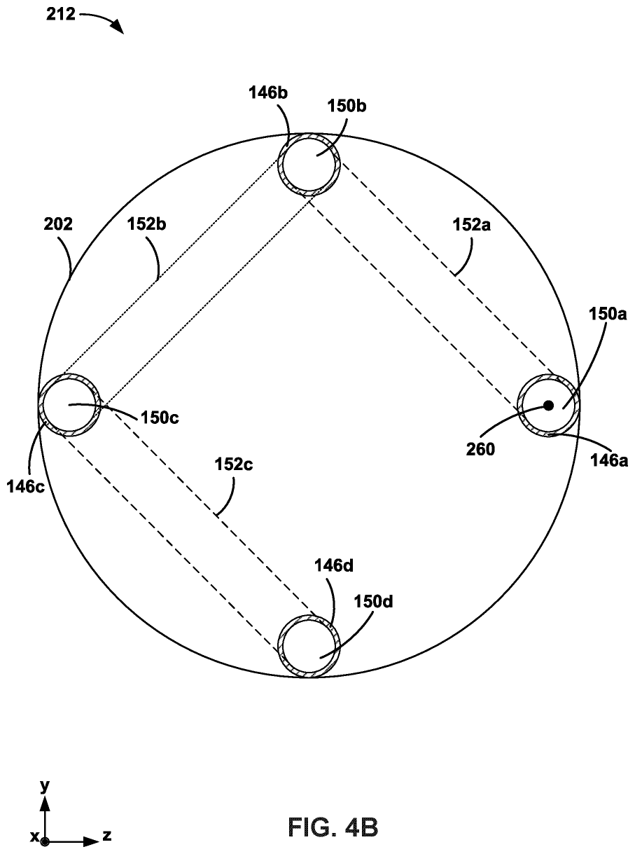

Neuromodulation element 112 may include a plurality of electrodes 146 (e.g., two electrodes, three electrodes, four electrodes, or the like) disposed along a length of elongate structure 142. Referring to FIG. 3A, neuromodulation element 112 may be configured to position a plurality of electrodes 146 at a longitudinal position 156 along neuromodulation element 112 and at different circumferential positions at longitudinal position 156. In the example shown, the longitudinal direction is along the x-axis, and neuromodulation element 112 is configured to position four electrodes 146a-146d substantially at longitudinal position 156 with each of electrodes at four different circumferential positions, e.g., about 90 degrees from each other. For example, first bend 152a may be substantially in the x-y plane in the example shown, second bend 152b may be substantially in the x-z plane, and third bend 152c may be substantially in the x-y plane. In this way, each of segments 150 may be separated from each other in the y and z directions, and each electrode 146 disposed on segments 150 may be separated from each other in the y and z directions and positioned, with neuromodulation element 112 in the expanded/deployed configuration, at different circumferential positions at longitudinal position 156. In other examples, electrodes 146 may be separated from each other in circumferential position by more or less than 90 degrees, and neuromodulation element 112 may include more or fewer than four electrodes 146, e.g., two electrodes 146, or three electrodes 146, or five or more electrodes 146. In the example shown, each of bends 152 are substantially 90 degrees and segments 150 are substantially parallel, e.g., along the x-direction and/or the longitudinal direction as illustrated in the example shown in FIG. 4B. In other examples, one or more of bends 152 may be more or less than 90 degrees, e.g., such that segments 150 may be at an angle relative to each other, e.g., at differing angles relative to the x-axis.

FIG. 3C illustrates another example neuromodulation elements 162. Neuromodulation element 162 may be substantially similar to neuromodulation element 112 described herein, except that neuromodulation element 162 includes electrodes 166a, 166b, 166, 166c, collectively “electrodes 166.” Electrodes 166 may be disposed along a length of elongate structure 142, and neuromodulation element 162 may be configured to position electrodes 166 at a second longitudinal position 176 different from longitudinal position 156 and along neuromodulation element 162 and at different circumferential positions at longitudinal position 176. The circumferential position of electrodes 166 may be the same or different from electrodes 146. Neuromodulation element 162 may be configured to deliver RF energy, modulate activity of one or more nerves, denervate one or more nerves, deliver a treatment, or the like, at different longitudinal positions within a vessel or other lumen of the patient, e.g., without repositioning catheter 102 and/or neuromodulation element 162. In some examples, neuromodulation element 162 may include any number of electrodes at any number of longitudinal positions. In some examples, neuromodulation element 162 may include any number of electrodes any number of circumferential positions at each longitudinal position, e.g., by including any number of segments 150 and bends 152.

The shape of neuromodulation element 112, e.g., elongate structure 142 including segments 150 and bends 152, may be configured to be compressible in the delivery configuration, to expand to the deployed configuration, e.g., in which electrodes 146 are radially extended to be adjacent, contacting, and/or proximate to an inner surface of a vessel. Bends 152 may be configured to be atraumatic, e.g., to the vessel and/or tissue of the patient and/or other portions of neuromodulation catheter 102 and/or a delivery device, such as a delivery sheath. Bends 152 may be configured to enable a delivery device, such as a sheath, to compress neuromodulation element 112 from the deployed to the delivery configuration by advancing the delivery device in a longitudinal direction (e.g., distally or proximally) to contact one or more of bends 152 and cause bends 152 and neuromodulation element 112 to compress, e.g., into a lumen of the delivery device.

In the example shown, neuromodulation element 112 includes distal tip 154. Distal tip may include distal opening 158, and distal opening may be an opening to a lumen within elongate structure 142 and/or elongate shaft 108. In some examples, distal tip 154 may be radiopaque. In some examples, distal tip 154 may comprise gold. In some examples, distal tip may not include distal opening 158 and may close off and/or terminate the lumen within elongate structure 142 and/or elongate shaft 108, e.g., distal tip 154 may be a distal endcap of elongate structure 142.

In some examples, distal tip 154 may be proximal to the most distal bend 152, and in some examples distal tip 154 may be proximal to the most proximal bend 152, e.g., bend 152b in the example shown. For example, distal tip 154 may be configured to reduce and/or prevent distal tip 154 from engaging with a vessel of the patient during insertion and/or delivery of neuromodulation element 112 to a target treatment site. In other examples, distal tip 154 may be distal to the most proximal bend 152, and in some examples distal tip may be distal to the most distal bend 152.

FIG. 4A is a cut-away of the view of an enlarged profile view of portions of another example neuromodulation element 212 in a deployed configuration, and FIG. 4B is a cross-sectional view of neuromodulation element 212 taken along longitudinal position 156 of FIG. 4A and in a deployed configuration within a vessel 202. Neuromodulation element 212 may be substantially similar to neuromodulation element 112 described above, except that neuromodulation element 212 includes guidewire port 262. FIG. 4A illustrates a view of lumen 242 within elongate structure 142 and guidewire 260 received within at least a portion of lumen 242.

In some examples, neuromodulation element 212 is configured to be navigated through vasculature of the patient, e.g., via guidewire 260. Neuromodulation element 212 includes guidewire port 262 configured to receive guidewire 260, e.g., into and/or out of lumen 242. For example, it may be difficult to guide and/or navigate neuromodulation element 212 via a guidewire within the entire length of elongate structure 142 including at least one bend 152. In the examples shown, neuromodulation element 212 may be navigated via guidewire 260 where guidewire 260 is within lumen 242 within a portion of elongate structure 142, e.g., without guidewire 260 having to traverse and/or follow the entire length of lumen 242 within elongate structure 142. In some examples, neuromodulation catheter 102 is configured to be advanced and/or retracted along guidewire 260 extending within elongate shaft 108 from proximal portion 108a of elongate shaft 108 to guidewire port 262 at the first bend 152a, e.g., via distal portion 108b of elongate shaft 108 and within segment 150a as illustrated in FIG. 4. In some examples, guidewire 260 may extend within elongate shaft 108 from a rapid exchange joint distal to proximal hypotube 128 (FIG. 2) to guidewire port 262 at the first bend 152a.

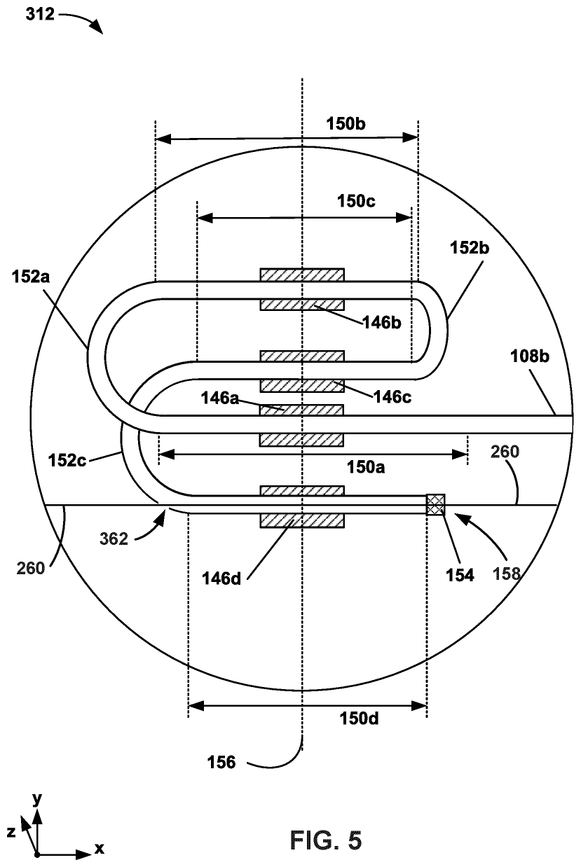

FIG. 5 is a cut-away of the view of an enlarged profile view of portions of another example neuromodulation element 312 in a deployed configuration. Neuromodulation element 312 may be substantially similar to neuromodulation element 212 described above, except that neuromodulation element 312 includes guidewire port 362 at the third bend 152c. In the examples shown, neuromodulation element 312 is configured to be advanced and/or retracted along guidewire 260 extending within lumen 242 between guidewire port 362 and distal opening 158 at distal tip 154, e.g., within segment 150d.

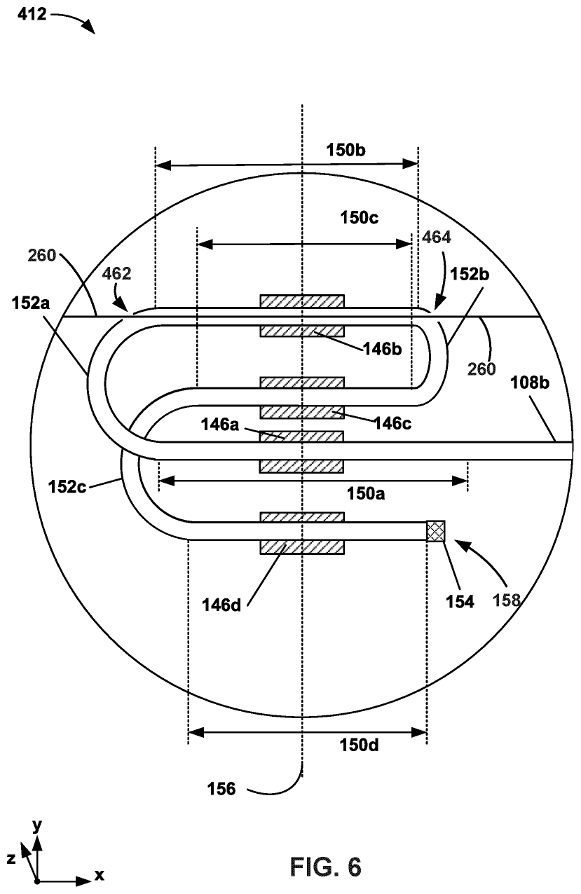

FIG. 6 is a cut-away of the view of an enlarged profile view of portions of another example neuromodulation element 412 in a deployed configuration. Neuromodulation element 412 may be substantially similar to neuromodulation element 212 described above, except that neuromodulation element 412 includes guidewire port 462 at the first bend 152a and guidewire port 464 at second bend 152b. In the examples shown, neuromodulation element 412 is configured to be advanced and/or retracted along guidewire 260 extending within lumen 242 between guidewire port 462 and guidewire port 464, e.g., within segment 150b.

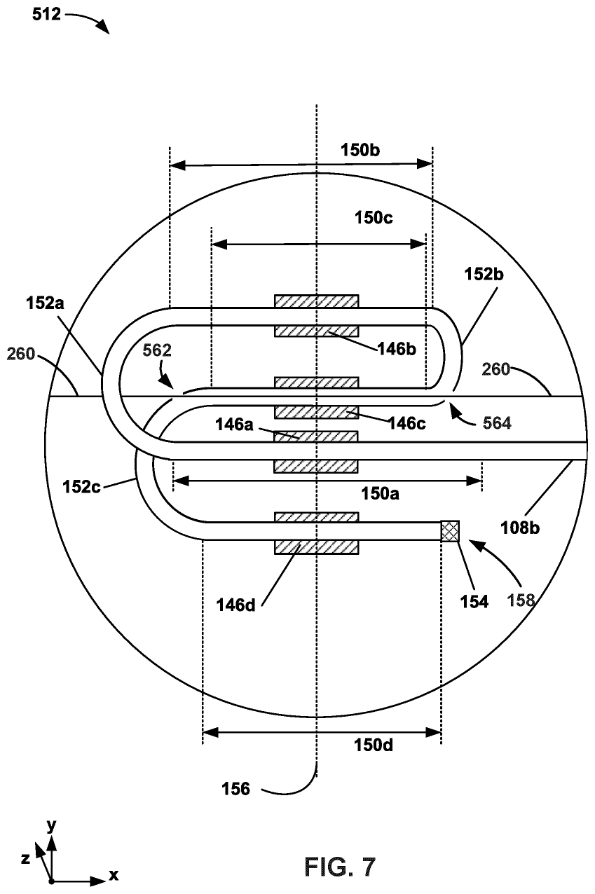

FIG. 7 is a cut-away of the view of an enlarged profile view of portions of another example neuromodulation element 512 in a deployed configuration. Neuromodulation element 512 may be substantially similar to neuromodulation element 212 described above, except that neuromodulation element 512 includes guidewire port 562 at the third bend 152c and guidewire port 564 at second bend 152b. In the examples shown, neuromodulation element 512 is configured to be advanced and/or retracted along guidewire 260 extending within lumen 242 between guidewire port 562 and guidewire port 564, e.g., within segment 150c.

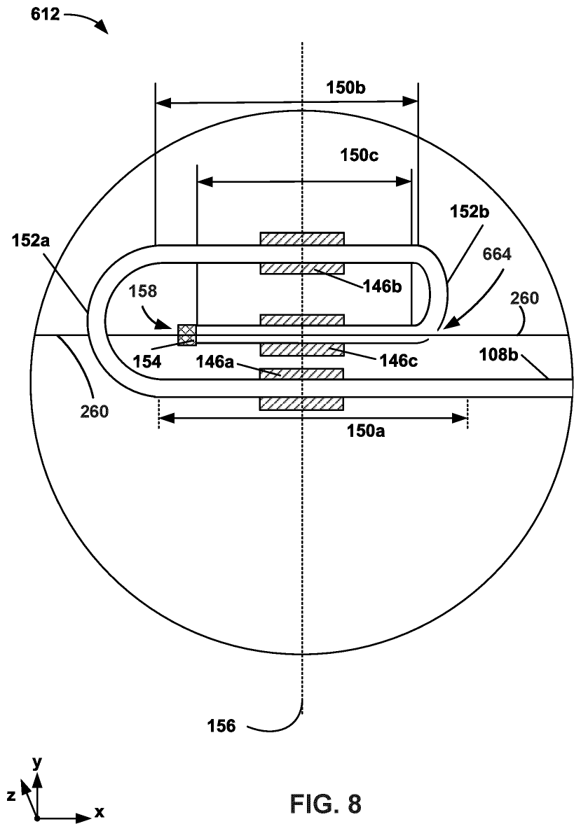

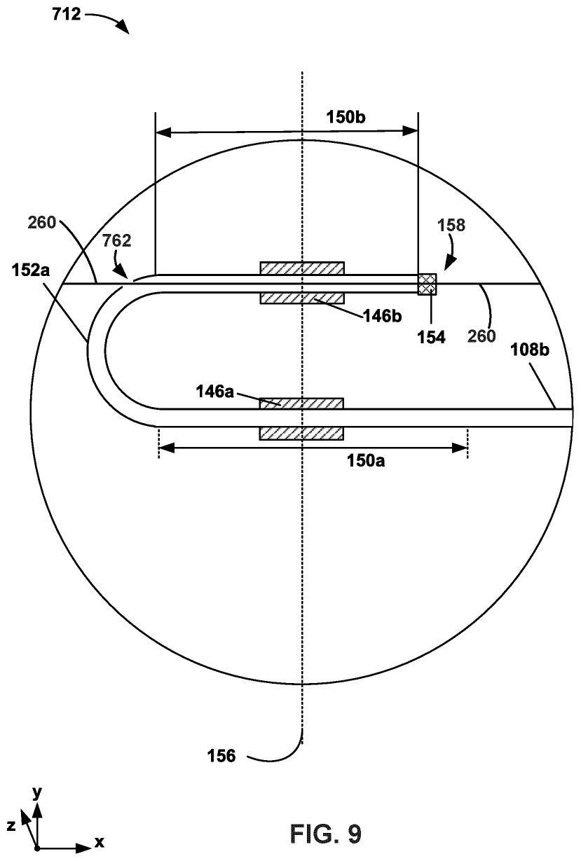

FIGS. 2-7 illustrate example neuromodulation elements including multiple bends and/or segments of elongate structure 142. In other examples, neuromodulation element may including more or fewer bends, more or fewer segments 150, and/or more or fewer electrodes 146. In some examples, neuromodulation elements 212, 312, 412, and 512 may include one or more guidewire ports located on any or all of bends 152 and/or segments 150, and may be configured to be advanced and/or retracted along guidewire 260 extending within lumen 242 within any of segments 150. FIGS. 8 and 9 illustrate example neuromodulation elements including fewer bends 152, segments 150, and electrodes 146 relative to FIGS. 2-7.

FIG. 8 is a cut-away of the view of an enlarged profile view of portions of another example neuromodulation element 612 in a deployed configuration. Neuromodulation element 612 may be substantially similar to neuromodulation element 212 described above, except that neuromodulation element 612 includes first bend 152a and second bend 152b, segments 150a, 150b, and 150c, electrodes 146a, 146b, and 146c, and guidewire port 664 at the second bend 152b. In the examples shown, neuromodulation element 612 is configured to be advanced and/or retracted along guidewire 260 extending within lumen 242 between guidewire port 664 and distal opening 158, e.g., within segment 150c. In some examples, neuromodulation element 612 may include one or more guidewire ports located on any or all of bends 152 and/or segments 150, and may be configured to be advanced and/or retracted along guidewire 260 extending within lumen 242 within any of segments 150.

Neuromodulation element 612 includes three electrodes 146 and elongate structure 142 may be configured to position electrodes 146 at different circumferential positions at longitudinal position 156 in the deployed configuration, e.g., with electrodes 146 circumferentially equally separated by about 120 degrees or any other separation.

FIG. 9 is a cut-away of the view of an enlarged profile view of portions of another example neuromodulation element 712 in a deployed configuration. Neuromodulation element 712 may be substantially similar to neuromodulation element 212 described above, except that neuromodulation element 712 includes first bend 152a, segments 150a and 150b, electrodes 146a and 146b, and guidewire port 762 at the first bend 152a. In the examples shown, neuromodulation element 712 is configured to be advanced and/or retracted along guidewire 260 extending within lumen 242 between guidewire port 762 and distal opening 158, e.g., within segment 150b. Neuromodulation element 712 includes two electrodes 146 and elongate structure 142 may be configured to position electrodes 146 at different circumferential positions at longitudinal position 156 in the deployed configuration, e.g., with electrodes 146 circumferentially equally separated by about 180 degrees or any other separation. In some examples, neuromodulation element 712 may include one or more guidewire ports located on any or all of bends 152 and may be configured to be advanced and/or retracted along guidewire 260 extending within lumen 242 within any of segments 150.

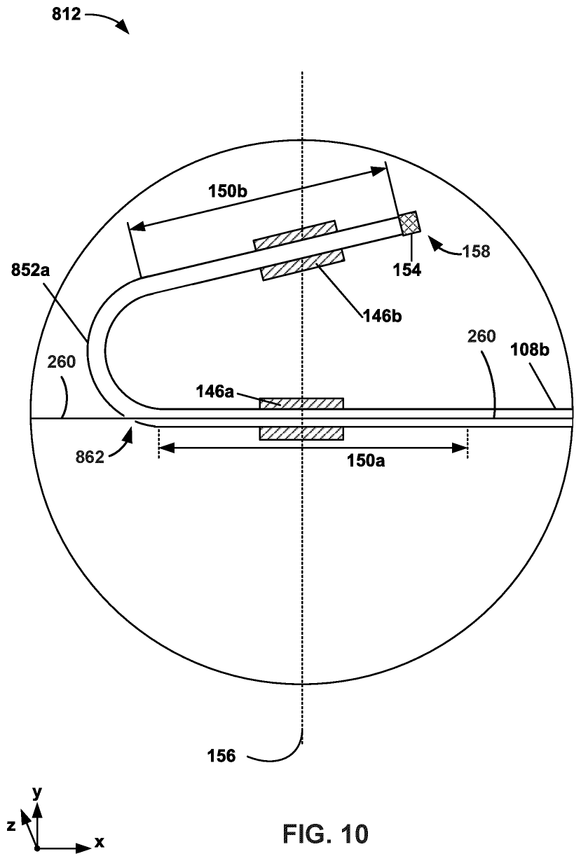

FIG. 10 is a cut-away of the view of an enlarged exploded profile view of portions of another example neuromodulation element 812 in a deployed configuration. Neuromodulation element 812 may be substantially similar to neuromodulation element 212 described above, except that neuromodulation element 812 includes first bend 852a different from first bend 152a. First bend 852a is configured to position segments 150a and 150b at an angle relative to each other, e.g., in the x-y plane in the example shown. In other examples, first bend 852a may be configured to position segments 150a and 150b and an angle relative to each other in the x-z plane, or in both the x-y and x-z planes. Neuromodulation element 812 also includes electrodes 146a and 146b and guidewire port 862 at the first bend 152a. In the examples shown, neuromodulation element 812 is configured to be advanced and/or retracted along guidewire 260 extending within elongate shaft 108 from proximal portion 108a of elongate shaft 108 to guidewire port 862 at the first bend 852a, e.g., via distal portion 108b of elongate shaft 108 and within segment 150a as illustrated in FIG. 10.

Neuromodulation elements 612, 712, and 812 of FIGS. 8-10 are example neuromodulation elements having fewer segments and/or bends than, for example, neuromodulation elements 112, 162, 212, 312, 412, and 512 of FIGS. 2-7. The fewer segments and/or bends of neuromodulation elements 612-812 may be more suitable for different patient lumen (e.g., vessel) sizes. For example, neuromodulation elements 612-812 may be more suitable for smaller vessel sizes and neuromodulation elements 112-512 may be more suitable for larger vessel sizes. Additionally, neuromodulation elements 612-812 may be more suitable for different anatomical configurations, for example, in more tortuous vessel branches, and neuromodulation elements 112-512 may be more suitable for substantially straight vessel sections. Neuromodulation elements 112-512, having relatively more bends and/or segments than neuromodulation elements 612-812, may support more electrodes in the same circumferential plane and may be configured to make smaller, more targeted lesions which may be appropriate in complex anatomy. Neuromodulation elements 612-812, having relatively fewer bends, segments, and/or electrodes than neuromodulation elements 112-512, may include relatively electrodes to make relatively larger lesions, and in some examples, may consume relatively less electrical power, e.g., with fewer electrodes.

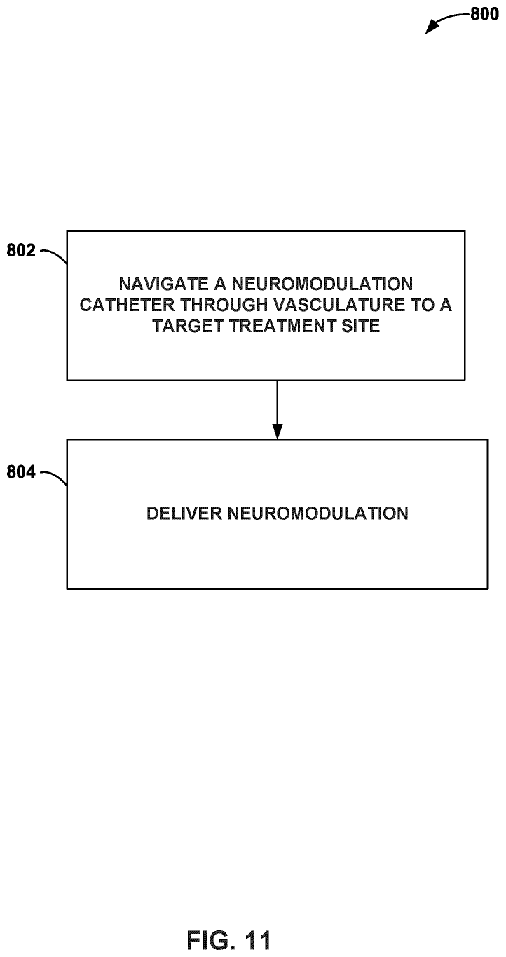

FIG. 11 is a flow chart illustrating an example method 800 for using a neuromodulation catheter 102, in accordance with one or more examples of the present disclosure. For ease of description, the example of FIG. 11 is described with regard to catheter 102 and any of neuromodulation elements 112-712 of FIGS. 2-10. However, such an example technique may be employed with any suitable catheter.

As shown in FIG. 11, a clinician or other user may navigate distal portion 108b of neuromodulation catheter 102 through vasculature of a patient to a target treatment site in a vessel of the patient (802). For example, the clinician may advance and/or retract neuromodulation catheter 102 and neuromodulation element 212 along a guidewire configured to enter or exit a lumen of the elongate structure at guidewire port 262 at first bend 152a. In some examples, the clinician may advance and/or retract neuromodulation catheter 102 and neuromodulation element 212 along a guidewire configured to enter or exit a lumen of the elongate structure at guidewire port 462 at first bend 152a and guidewire port 464 at second bend 152b. In some examples, the clinician may advance and/or retract neuromodulation catheter 102 and neuromodulation element 212 along a guidewire configured to enter or exit a lumen of the elongate structure at guidewire port 362 at bend 152d and distal opening 158.

The clinician or other user may cause neuromodulation catheter 102 to deliver neuromodulation to the target treatment site (804). For example, the clinician or user may cause neuromodulation element 112 to deploy to the radially expanded deployed state at or near the target treatment site. The clinician or user may then cause electrodes 146 to deliver energy to tissue of the patient at or near the target treatment site.

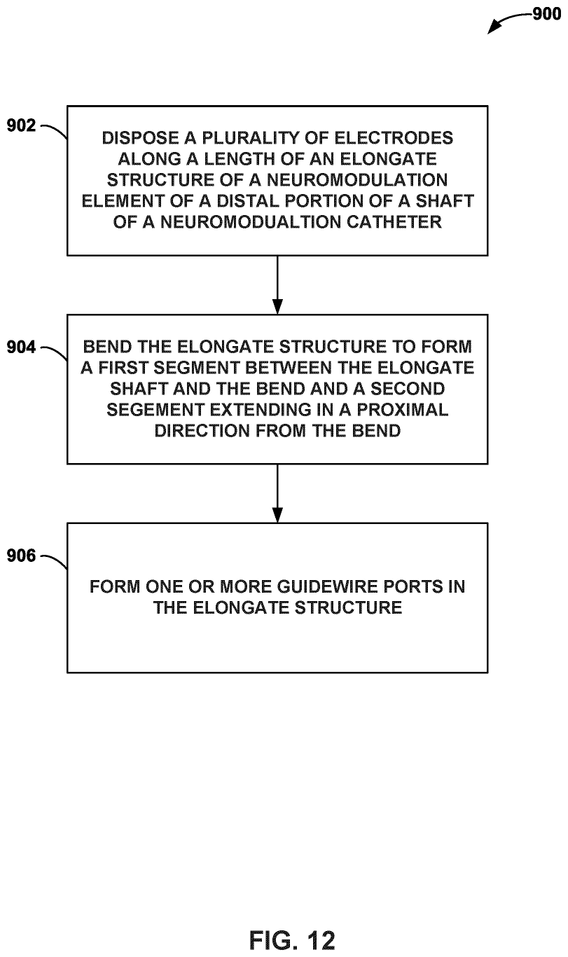

FIG. 12 is a flow chart illustrating an example method 900 for assembling components of a neuromodulation catheter, in accordance with one or more examples of the present disclosure. For ease of description, the example of FIG. 12 is described with regard to catheter 102 and any of neuromodulation elements 112-712 of FIGS. 2-10. However, such an example technique may be employed to assemble any suitable catheter.

Electrodes 146 may be disposed along a length of elongate structure 142 of neuromodulation element 212 of distal portion 108b of an elongate shaft 108 of neuromodulation catheter 102 (902). In some examples, at least one electrode may be disposed at, or proximate to, a distal tip of neuromodulation element 212, e.g., elongate structure 142. In some examples, a radiopaque indicator may be disposed along elongate structure 142, e.g., at the distal tip. In some examples, an electrode may be disposed at the distal tip 154 that is radiopaque, and may comprise gold.

Elongate structure 142 may be bent and/or curved to form a bend such that a first segment of the length of elongate structure 142 extends between the bend and elongate shaft 108 and a second segment of the length of the elongate structure 142 extends in a proximal direction from the bend (904). In some examples, elongate structure 142 may be bent via a cylindrical bend. In some examples, a plurality of electrodes 146 may be disposed on segments 150 before or after bending elongate structure 142, and electrodes 146 may be disposed, and/or elongate structure 142 may be bent, such that the electrodes 146 are positioned at the same longitudinal position, e.g., longitudinal position 156 when neuromodulation element 212 is in a deployed configuration. In some examples, elongate structure 142 may be bent such that at least one of the segments of elongate structure 142 formed by one or more bends is substantially parallel with one or more of the other segments. In other examples, elongate structure 142 may be bent such that at least one of the segments of elongate structure 142 formed by one or more bends is at an angle relative to one or more of the other segments. In some examples, one or more of electrodes 146 may be disposed on one or more of segments 150 formed by bending elongate structure 142 with one or more bends 152.

In some examples, one or more guidewire ports may be formed at one or more bends and/or segments of elongate structure 142 (906). The one or more guidewire ports may be formed to receive a guidewire, e.g., into and/or out of lumen 242 of elongate structure 142.

Catheters configured in accordance with at least some examples of the present technology may be well suited (e.g., with respect to sizing, flexibility, operational characteristics, and/or other attributes) for performing renal neuromodulation in human patients. Renal neuromodulation is the partial or complete incapacitation or other effective disruption of nerves of the kidneys (e.g., nerves terminating in the kidneys or in structures closely associated with the kidneys). In particular, renal neuromodulation may include inhibiting, reducing, and/or blocking neural communication along neural fibers (e.g., efferent and/or afferent neural fibers) of the kidneys. Such incapacitation may be long-term (e.g., permanent or for periods of months, years, or decades) or short-term (e.g., for periods of minutes, hours, days, or weeks). Renal neuromodulation is expected to contribute to the systemic reduction of sympathetic tone or drive and/or to benefit at least some specific organs and/or other bodily structures innervated by sympathetic nerves. Accordingly, renal neuromodulation is expected to be useful in treating clinical conditions associated with systemic sympathetic overactivity or hyperactivity, particularly conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation may be electrically-induced, thermally-induced, or induced in another suitable manner or combination of manners at one or more suitable treatment locations during a treatment procedure. The treatment location may be within or otherwise proximate to a renal lumen (e.g., a renal artery, a ureter, a renal pelvis, a major renal calyx, a minor renal calyx, or another suitable structure), and the treated tissue may include tissue at least proximate to a wall of the renal lumen. For example, with regard to a renal artery, a treatment procedure may include modulating nerves in the renal plexus, which lay intimately within or adjacent to the adventitia of the renal artery. Various suitable modifications may be made to the catheters described above to accommodate different treatment modalities. For example, the band electrodes 204 may be replaced with transducers to facilitate transducer-based treatment modalities.

Renal neuromodulation may include an electrode-based treatment modality alone or in combination with another treatment modality. Electrode-based treatment may include delivering electricity and/or another form of energy to tissue at or near a treatment location to stimulate and/or heat the tissue in a manner that modulates neural function. For example, sufficiently stimulating and/or heating at least a portion of a sympathetic renal nerve may slow or potentially block conduction of neural signals to produce a prolonged or permanent reduction in renal sympathetic activity. A variety of suitable types of energy may be used to stimulate and/or heat tissue at or near a treatment location. For example, neuromodulation in accordance with examples of the present technology may include delivering RF energy and/or another suitable type of energy. An electrode used to deliver this energy may be used alone or with other electrodes in a multi-electrode array.

Heating effects of electrode-based treatment may include ablation and/or non-ablative alteration or damage (e.g., via sustained heating and/or resistive heating). For example, a treatment procedure may include raising the temperature of target neural fibers to a target temperature above a first threshold to achieve non-ablative alteration, or above a second, higher threshold to achieve ablation. The target temperature may be higher than about body temperature (e.g., about 37° C.) but less than about 45° C. for non-ablative alteration, and the target temperature may be higher than about 45° C. for ablation. Heating tissue to a temperature between about body temperature and about 45° C. may induce non-ablative alteration, for example, via moderate heating of target neural fibers or of luminal structures that perfuse the target neural fibers. In cases where luminal structures are affected, the target neural fibers may be denied perfusion resulting in necrosis of the neural tissue. Heating tissue to a target temperature higher than about 45° C. (e.g., higher than about 60° C.) may induce ablation, for example, via substantial heating of target neural fibers or of luminal structures that perfuse the target fibers. In some patients, it may be desirable to heat tissue to temperatures that are sufficient to ablate the target neural fibers or the luminal structures, but that are less than about 90° C. (e.g., less than about 85° C., less than about 80° C., or less than about 75° C.).
Clause 1: A catheter comprising: an elongate shaft including a proximal portion and a distal portion including a neuromodulation element, wherein the neuromodulation element comprises: a plurality of electrodes disposed along a length of an elongate structure; and a bend between a first segment of the length of the elongate structure and second segment of the length of the elongate structure such that the second segment extends in a proximal direction from the bend.
Clause 2: The catheter of clause 1, wherein the elongate structure further comprises a guidewire port at the bend, and wherein the guidewire port is configured to receive a guidewire.
Clause 3: The catheter of clause 2, wherein the catheter is configured to be at least one of advanced or retracted along the guidewire extending within the elongate shaft from the proximal portion of the elongate shaft to the guidewire port.
Clause 4: The catheter of clause 2, wherein the bend is a first bend, wherein the elongate structure comprises a second bend such that a third segment of the length of the elongate structure extends in a distal direction from the second bend.
Clause 5: The catheter of clause 4, wherein the guidewire port is a first guidewire port, wherein the elongate structure further comprises a second guidewire port at the second bend, and wherein the second guidewire port is configured to receive a guidewire.
Clause 6: The catheter of clause 5, wherein the catheter is configured to be at least one of advanced or retracted along the guidewire extending within the elongate structure from the guidewire port to a distal opening proximate a distal tip of the elongate shaft.
Clause 7: The catheter of clause 4, wherein the elongate structure comprises a third bend such that a fourth segment of the length of the elongate structure extends in a proximal direction from the third bend.
Clause 8: The catheter of clause 7, wherein the guidewire port is a first guidewire port, wherein the elongate structure further comprises a second guidewire port at the second bend, and wherein the second guidewire port is configured to receive a guidewire
Clause 9: The catheter of clause 8, wherein the catheter is configured to be at least one of advanced or retracted along the guidewire extending within the elongate structure from the first guidewire port to the second guidewire port.
Clause 10: The catheter of any one of clauses 1 through 9, wherein a first electrode of the plurality of electrodes is disposed along the first segment and a second electrode of the plurality of electrodes is disposed along the second segment.
Clause 11: The catheter of clause 10, wherein the first electrode and the second electrode are disposed at a first longitudinal position along the elongate structure.
Clause 12: The catheter of clause 11, wherein the first electrode and the second electrode are configured to provide a neuromodulation treatment to tissue of the patient at the first longitudinal position and at different circumferential positions within the vasculature of the patient.
Clause 13: The catheter of clause 11 or clause 12, wherein the bend is a first bend, wherein the elongate structure comprises a second bend such that a third segment of the length of the elongate structure extends in a distal direction from the second bend, wherein a third electrode of the plurality of neuro modulation elements is disposed along the second segment.
Clause 14: The catheter of clause 13, wherein the third electrode is disposed at the first longitudinal position along elongate structure.
Clause 15: The catheter of clause 14, wherein the third electrode is configured to provide a neuromodulation treatment to tissue of the patient at the first longitudinal position and at a circumferential position within the vasculature of the patient different from the circumferential position of the first electrode and the circumferential position of the second electrode.
Clause 16: The catheter of clause 14 or clause 15, wherein the elongate structure comprises a third bend such that a fourth segment of the length of the elongate structure extends in a proximal direction from the third bend, wherein a fourth electrode of the plurality of electrodes is disposed along the fourth segment.
Clause 17: The catheter of clause 16, wherein the fourth neuromodulation element is disposed at the first longitudinal position along elongate structure.
Clause 18: The catheter of clause 17, wherein the fourth electrode is configured to provide a neuromodulation treatment to tissue of the patient at the first longitudinal position and at a circumferential position within the vasculature of the patient different from the circumferential position of any of the first, second, and third electrodes.
Clause 19: The catheter of any one of clauses 1 through 18, further comprising a distal electrode disposed at the distal tip of the elongate structure.
Clause 20: The catheter of any one of clauses 1 through 19, wherein a distal tip of the elongate structure is configured to be radiopaque.
Clause 21: The catheter of any one of clauses 1 through 20, wherein a distal tip of the elongate structure comprises gold.
Clause 22: The catheter of any one of clauses 1 through 21, wherein the bend comprises a cylindrical bend.
Clause 23: The catheter of any one of clauses 1 through 22, wherein the first segment is at an angle relative to the second segment.
Clause 24: A method comprising: navigating a catheter through vasculature of a patient to a target treatment site in a vessel of the patient, wherein the catheter comprises an elongate shaft including a proximal portion and a distal portion including a neuromodulation element, wherein the neuromodulation element comprises: a plurality of electrodes disposed along a length of an elongate structure; and a bend between a first segment of the length of the elongate structure and second segment of the length of the elongate structure such that the second segment extends in a proximal direction from the bend.
Clause 25: The method of clause 24, wherein navigating the catheter comprises at least one of advancing or retracting the neuromodulation element along a guidewire configured to enter or exit a lumen of the elongate structure at a first guidewire port at the bend.
Clause 26: The method of clause 24, wherein the bend is a first bend, wherein the elongate structure comprises a second bend such that a third segment of the length of the elongate structure extends in a distal direction from the second bend, wherein navigating the catheter comprises at least one of advancing or retracting the neuromodulation element along a guidewire configured to enter or exit a lumen of the elongate structure at a second guidewire port at the second bend.
Clause 27: The method of clause 26, wherein the elongate structure comprises a third bend such that a fourth segment of the length of the elongate structure extends in a proximal direction from the third bend, wherein navigating the catheter comprises at least one of advancing or retracting the neuromodulation element along a guidewire configured to enter or exit the lumen of the elongate structure at a third guidewire port at the third bend.
Clause 28: A method comprising: disposing a plurality of electrodes along a length of an elongate structure of a neuromodulation element of distal portion of an elongate shaft of a catheter; and bending the elongate structure to form a bend such that a first segment of the length of the elongate structure extends between the bend and the elongate shaft and a second segment of the length of the elongate structure extends in a proximal direction from the bend.
Clause 29: The method of clause 28, further comprising forming a guidewire port at the bend, wherein the guidewire port is configured to receive a guidewire.
Clause 30: The method of clause 28 or clause 29, wherein the bend is a first bend, the method further comprising bending the elongate structure to form a second bend such that a third segment of the length of the elongate structure extends in a distal direction from the second bend.
Clause 31: The method of clause 30, further comprising forming a guidewire port at the second bend, wherein the guidewire port is configured to receive the guidewire.
Clause 32: The method of clause 30 or clause 31, further comprising bending the distal portion to form a third bend such that a fourth segment of the length of the elongate structure extends in a proximal direction from the third bend.
Clause 33: The method of clause 32, wherein the guidewire port is a first guidewire port, the method further comprising forming a second guidewire port at the third bend, wherein the second guidewire port is configured to receive the guidewire.
Clause 34: The method of clause 32 or clause 33, wherein disposing the plurality of electrodes along the length of the elongate structure comprises disposing at least one electrode along at least one of the first segment, the second segment, the third segment, or the fourth segment.
Clause 35: The method of any one of clauses 32 through 34, wherein at least one of the first segment, the second segment, or the third segment is formed to be substantially parallel with at least one other of the first segment, the second segment, or the third segment.
Clause 36: The method of any one of clauses 28 through 35, wherein disposing the plurality of electrodes along the length of the elongate structure comprises disposing at least one electrode at a distal tip of the neuromodulation element.
Clause 37: The method of any one of clauses 28 through 36, further comprising disposing a radiopaque indicator at a distal tip of the neuromodulation element.
Clause 38: The method of any one of clauses 28 through 37, wherein a distal tip of the neuromodulation element comprises gold.
Clause 39: The method of any one of clauses 28 through 38, wherein bending the elongate structure comprises cylindrically bending the elongate structure.

This disclosure is not intended to be exhaustive or to limit the present technology to the precise forms disclosed herein. Although specific examples are disclosed herein for illustrative purposes, various equivalent modifications are possible without deviating from the present technology, as those of ordinary skill in the relevant art will recognize. In some cases, well-known structures and functions have not been shown and/or described in detail to avoid unnecessarily obscuring the description of the examples of the present technology. Although steps of methods may be presented herein in a particular order, in alternative examples the steps may have another suitable order. Similarly, certain aspects of the present technology disclosed in the context of particular examples may be combined or eliminated in other examples. Furthermore, while advantages associated with certain examples may have been disclosed in the context of those examples, other examples may also exhibit such advantages, and not all examples need necessarily exhibit such advantages or other advantages disclosed herein to fall within the scope of the present technology. Accordingly, this disclosure and associated technology may encompass other examples not expressly shown and/or described herein.

The methods disclosed herein include and encompass, in addition to methods of practicing the present technology (e.g., methods of making and using the disclosed devices and systems), methods of instructing others to practice the present technology.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout this disclosure to mean including at least the recited feature(s) such that any greater number of the same feature(s) and/or one or more additional types of features are not precluded. Directional terms, such as “upper,” “lower,” “front,” “back,” “vertical,” and “horizontal,” may be used herein to express and clarify the relationship between various elements. It should be understood that such terms do not denote absolute orientation. Reference herein to “one example,” “an example,” or similar formulations means that a particular feature, structure, operation, or characteristic described in connection with the example may be included in at least one example of the present technology. Thus, the appearances of such phrases or formulations herein are not necessarily all referring to the same example. Furthermore, various particular features, structures, operations, or characteristics may be combined in any suitable manner in one or more examples of the present technology.

Various aspects of the disclosure have been described. These and other aspects are within the scope of the following claims.

Further disclosed herein is the subject-matter of the following clauses:
1. A catheter comprising:
   an elongate shaft including a proximal portion and a distal portion including a neuromodulation element, wherein the neuromodulation element comprises:
      a plurality of electrodes disposed along a length of an elongate structure; and
      a bend between a first segment of the length of the elongate structure and second segment of the length of the elongate structure such that the second segment extends in a proximal direction from the bend.
2. The catheter of clause 1, wherein the elongate structure further comprises a guidewire port at the bend, and wherein the guidewire port is configured to receive a guidewire.
3. The catheter of clause 2, wherein the catheter is configured to be at least one of advanced or retracted along the guidewire extending within the elongate shaft from the proximal portion of the elongate shaft to the guidewire port; or wherein the bend is a first bend and the elongate structure comprises a second bend such that a third segment of the length of the elongate structure extends in a distal direction from the second bend.
4. The catheter of clause 3, wherein the guidewire port is a first guidewire port, wherein the elongate structure further comprises a second guidewire port at the second bend, and wherein the second guidewire port is configured to receive a guidewire.
5. The catheter of clause 3, wherein the elongate structure comprises a third bend such that a fourth segment of the length of the elongate structure extends in a proximal direction from the third bend.
6. The catheter of clause 5, wherein the guidewire port is a first guidewire port, wherein the elongate structure further comprises a second guidewire port at the second bend, and wherein the second guidewire port is configured to receive a guidewire.
7. The catheter of any one of clauses 1 through 6, wherein a first electrode of the plurality of electrodes is disposed along the first segment and a second electrode of the plurality of electrodes is disposed along the second segment.
8. The catheter of clause 7, wherein the first electrode and the second electrode are disposed at a first longitudinal position along the elongate structure.
9. The catheter of clause 8, wherein the first electrode and the second electrode are configured to provide a neuromodulation treatment to tissue of the patient at the first longitudinal position and at different circumferential positions within the vasculature of the patient.
10. The catheter of clause 8 or clause 9, wherein the bend is a first bend, wherein the elongate structure comprises a second bend such that a third segment of the length of the elongate structure extends in a distal direction from the second bend, wherein a third electrode of the plurality of neuro modulation elements is disposed along the second segment.
11. The catheter of clause 10, wherein the third electrode is disposed at the first longitudinal position along elongate structure.
12. The catheter of clause 11, wherein the third electrode is configured to provide a neuromodulation treatment to tissue of the patient at the first longitudinal position and at a circumferential position within the vasculature of the patient different from the circumferential position of the first electrode and the circumferential position of the second electrode.
13. The catheter of clause 11 or clause 12, wherein the elongate structure comprises a third bend such that a fourth segment of the length of the elongate structure extends in a proximal direction from the third bend, wherein a fourth electrode of the plurality of electrodes is disposed along the fourth segment.
14. The catheter of clause 13, wherein the fourth neuromodulation element is disposed at the first longitudinal position along elongate structure.
15. The catheter of clause 14, wherein the fourth electrode is configured to provide a neuromodulation treatment to tissue of the patient at the first longitudinal position and at a circumferential position within the vasculature of the patient different from the circumferential position of any of the first, second, and third electrodes.
16. The catheter of any one of clauses 1 through 15, further comprising a distal electrode disposed at the distal tip of the elongate structure.
17. The catheter of any one of clauses 1 through 16, wherein a distal tip of the elongate structure is radiopaque.
18. The catheter of any one of clauses 1 through 17, wherein a distal tip of the elongate structure comprises gold.
19. A method comprising:
   navigating a catheter through vasculature of a patient to a target treatment site in a vessel of the patient, wherein the catheter comprises an elongate shaft including a proximal portion and a distal portion including a neuromodulation element, wherein the neuromodulation element comprises:
   a plurality of electrodes disposed along a length of an elongate structure; and
   a bend between a first segment of the length of the elongate structure and second segment of the length of the elongate structure such that the second segment extends in a proximal direction from the bend.
20. The method of clause 19, wherein navigating the catheter comprises at least one of advancing or retracting the neuromodulation element along a guidewire configured to enter or exit a lumen of the elongate structure at a first guidewire port at the bend.
21. A method comprising:
   disposing a plurality of electrodes along a length of an elongate structure of a neuromodulation element of distal portion of an elongate shaft of a catheter; and
   bending the elongate structure to form a bend such that a first segment of the length of the elongate structure extends between the bend and the elongate shaft and a second segment of the length of the elongate structure extends in a proximal direction from the bend.
22. The method of clause 21, further comprising forming a guidewire port at the bend, wherein the guidewire port is configured to receive a guidewire.
23. The method of clause 21 or clause 22, wherein the bend is a first bend, the method further comprising bending the elongate structure to form a second bend such that a third segment of the length of the elongate structure extends in a distal direction from the second bend.
24. The method of clause 23, further comprising forming a guidewire port at the second bend, wherein the guidewire port is configured to receive the guidewire.
25. The method of clause 23 or clause 24, further comprising bending the distal portion to form a third bend such that a fourth segment of the length of the elongate structure extends in a proximal direction from the third bend.
26. The method of clause 25, wherein the guidewire port is a first guidewire port, the method further comprising forming a second guidewire port at the third bend, wherein the second guidewire port is configured to receive the guidewire.

1. A catheter comprising:
an elongate shaft including a proximal portion and a distal portion including a neuromodulation element, wherein the neuromodulation element comprises:
a plurality of electrodes disposed along a length of an elongate structure; and
a bend between a first segment of the length of the elongate structure and second segment of the length of the elongate structure such that the second segment extends in a proximal direction from the bend.

2. The catheter of claim 1, wherein the elongate structure further comprises a guidewire port at the bend, and wherein the guidewire port is configured to receive a guidewire.

3. The catheter of claim 2, wherein the catheter is configured to be at least one of advanced or retracted along the guidewire extending within the elongate shaft from the proximal portion of the elongate shaft to the guidewire port; or
wherein the bend is a first bend and the elongate structure comprises a second bend such that a third segment of the length of the elongate structure extends in a distal direction from the second bend.

4. The catheter of claim 3, wherein the guidewire port is a first guidewire port, wherein the elongate structure further comprises a second guidewire port at the second bend, and wherein the second guidewire port is configured to receive a guidewire.

5. The catheter of claim 3, wherein the elongate structure comprises a third bend such that a fourth segment of the length of the elongate structure extends in a proximal direction from the third bend.

6. The catheter of claim 5, wherein the guidewire port is a first guidewire port, wherein the elongate structure further comprises a second guidewire port at the second bend, and wherein the second guidewire port is configured to receive a guidewire.

7. The catheter of claim 1, wherein a first electrode of the plurality of electrodes is disposed along the first segment and a second electrode of the plurality of electrodes is disposed along the second segment.

8. The catheter of claim 7, wherein the first electrode and the second electrode are disposed at a first longitudinal position along the elongate structure.

9. The catheter of claim 8, wherein the first electrode and the second electrode are configured to provide a neuromodulation treatment to tissue of the patient at the first longitudinal position and at different circumferential positions within the vasculature of the patient.

10. The catheter of claim 8, wherein the bend is a first bend, wherein the elongate structure comprises a second bend such that a third segment of the length of the elongate structure extends in a distal direction from the second bend, wherein a third electrode of the plurality of neuro modulation elements is disposed along the second segment.

11. The catheter of claim 10, wherein the third electrode is disposed at the first longitudinal position along elongate structure.

12. The catheter of claim 11, wherein the third electrode is configured to provide a neuromodulation treatment to tissue of the patient at the first longitudinal position and at a circumferential position within the vasculature of the patient different from the circumferential position of the first electrode and the circumferential position of the second electrode.

13. The catheter of claim 11, wherein the elongate structure comprises a third bend such that a fourth segment of the length of the elongate structure extends in a proximal direction from the third bend, wherein a fourth electrode of the plurality of electrodes is disposed along the fourth segment.

14. The catheter of claim 13, wherein the fourth neuromodulation element is disposed at the first longitudinal position along elongate structure.

15. The catheter of claim 14, wherein the fourth electrode is configured to provide a neuromodulation treatment to tissue of the patient at the first longitudinal position and at a circumferential position within the vasculature of the patient different from the circumferential position of any of the first, second, and third electrodes.

16. The catheter of claim 1, further comprising a distal electrode disposed at the distal tip of the elongate structure.

17. The catheter of claim 1, wherein a distal tip of the elongate structure is radiopaque.

18. The catheter of claim 1, wherein a distal tip of the elongate structure comprises gold.

19. A method comprising:
navigating a catheter through vasculature of a patient to a target treatment site in a vessel of the patient, wherein the catheter comprises an elongate shaft including a proximal portion and a distal portion including a neuromodulation element, wherein the neuromodulation element comprises:
a plurality of electrodes disposed along a length of an elongate structure; and
a bend between a first segment of the length of the elongate structure and second segment of the length of the elongate structure such that the second segment extends in a proximal direction from the bend.

20. The method of claim 19, wherein navigating the catheter comprises at least one of advancing or retracting the neuromodulation element along a guidewire configured to enter or exit a lumen of the elongate structure at a first guidewire port at the bend.

21-26. (canceled)
